(19) United States
(12) Patent Application Publication
(10) Pub. No.: US 2026/0191843 A1
(43) Pub. Date: Jul. 9, 2026

(54) METHODS AND MATERIALS FOR TREATING BURNS

(71) Applicant: The Penn State Research Foundation, University Park, PA (US)

(72) Inventors: Jagadeeshaprasad Mashanipalya Guddadarangaiah, University Park, PA (US); Prem Kumar Govindappa, University Park, PA (US); Mosammat Begom, University Park, PA (US); John Elfar, Hummelstown, PA (US)

(21) Appl. No.: 19/445,897

(22) Filed: Jan. 12, 2026

Related U.S. Application Data
(63) Continuation of application No. 18/139,123, filed on Apr. 25, 2023, now abandoned.
(60) Provisional application No. 63/334,537, filed on Apr. 25, 2022.

(51) Int. Cl. A61K 31/4409; A61P 17/02

(52) U.S. Cl. CPC A61K 31/4409; A61P 17/02

(57) ABSTRACT

This document relates to methods and materials for treating burns. For example, compositions containing 4-aminopyridine (4-AP) and/or one or more derivatives of 4-AP can be administered (e.g., systemically administered) to a mammal having one or more burns to treat the burn(s) (e.g., to promote burn wound healing).

### CROSS-REFERENCE TO RELATED APPLICATIONS

This application is a continuation of U.S. application Ser. No. 18/139,123, filed Apr. 25, 2023, which claims the benefit of U.S. Patent Application Ser. No. 63/334,537, filed on Apr. 25, 2022. The disclosure of the prior application is considered part of (and are incorporated by reference in) the disclosure of this application.

### TECHNICAL FIELD

This document relates to methods and materials for treating burns. For example, compositions containing 4-aminopyridine (4-AP) and/or one or more derivatives of 4-AP can be administered (e.g., systemically administered) to a mammal having one or more burns to treat the burn(s) (e.g., to promote burn wound healing).

### BACKGROUND INFORMATION

Skin burn wounds are generally associated with an intensive imbalance of tissue fluids and electrolytes and delayed healing of the burned area with pathophysiological secondary complications (e.g., inflammation, metabolic disorders, catabolism of muscle tissue, and complications in vital organs). Burn wound complications often require the use of various medications to provide comprehensive life-or-limb-saving treatment. To date, many approaches such as tissue engineering and regenerative medicine may improve burn wound healing and restore skin physiology. However, most strategies have limited use due to the complexity of burns. Unaddressed problems in burn wound healing include necrosis, inflammation, delayed healing, and scarring.

### SUMMARY

This document provides methods and materials for treating burns. For example, compositions containing 4-AP and/or one or more derivatives of 4-AP can be used to treat a mammal (e.g., a human) having one or more burns. In some cases, a composition containing 4-AP and/or one or more derivatives of 4-AP can be administered (e.g., systemically administered) to a mammal (e.g., a human) having one or more burns to treat the mammal (e.g., promote burn wound healing).

As demonstrated herein, systemic administration of 4-AP can protect burn wounds from early necrosis and can accelerate burn wound healing. For example, administration of 4-AP can protect a burn wound from early necrosis and apoptosis of a burn skin wound at a zone of stasis. For example, administration of 4-AP can accelerate skin burn wound healing and skin regeneration. Having the ability to accelerate burn wound healing by administering (e.g., systemically administering) a composition containing 4-AP and/or one or more derivatives of 4-AP as described herein provides a unique and non-invasive way to treat burns.

In general, one aspect of this document features methods for treating a burn. The methods can include, or consist essentially of, administering a composition including 4-AP or one or more derivatives of 4-AP to a mammal identified as having a burn. The mammal can be a human. The burn can be a cutaneous burn. The burn can be a thermal burn, a flame burn, a scalding burn, a cold burn, a chemical burn, an electricity burn, an ionizing radiation burn, a non-ionizing radiation burn, or a sun burn. The administration can be a systemic administration. The administration can be a topical administration.

In another aspect, this document features methods for promoting burn wound healing. The methods can include, or consist essentially of, administering a composition including 4-AP or one or more derivatives of 4-AP to a mammal identified as having a burn, thereby promoting healing of the burn. The mammal can be a human. The burn can be a cutaneous burn. The burn can be a thermal burn, a flame burn, a scalding burn, a cold burn, a chemical burn, an electricity burn, an ionizing radiation burn, a non-ionizing radiation burn, or a sun burn. The administration can be a systemic administration. The administration can be a topical administration.

In another aspect, this document features methods for accelerating skin regeneration of a burn region. The methods can include, or consist essentially of, administering a composition including 4-AP or one or more derivatives of 4-AP to a mammal identified as having a burn, thereby accelerating skin regeneration of tissue around the burn. The mammal can be a human. The burn can be a cutaneous burn. The burn can be a thermal burn, a flame burn, a scalding burn, a cold burn, a chemical burn, an electricity burn, an ionizing radiation burn, a non-ionizing radiation burn, or a sun burn. The administration can be a systemic administration. The administration can be a topical administration.

In another aspect, this document features uses of a composition including 4-AP or one or more derivatives of 4-AP to treat a burn. The burn can be a cutaneous burn. The burn can be a thermal burn, a flame burn, a scalding burn, a cold burn, a chemical burn, an electricity burn, an ionizing radiation burn, a non-ionizing radiation burn, or a sun burn.

In another aspect, this document features compositions including 4-AP or one or more derivatives of 4-AP for use as a medicament to treat a burn. The burn can be a cutaneous burn. The burn can be a thermal burn, a flame burn, a scalding burn, a cold burn, a chemical burn, an electricity burn, an ionizing radiation burn, a non-ionizing radiation burn, or a sun burn.

In another aspect, this document features compositions including 4-AP or one or more derivatives of 4-AP for use in the treatment of a burn. The burn can be a cutaneous burn. The burn can be a thermal burn, a flame burn, a scalding burn, a cold burn, a chemical burn, an electricity burn, an ionizing radiation burn, a non-ionizing radiation burn, or a sun burn.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C show that 4-AP can promote burn wound closure and tissue regeneration. FIG. 1A) Representative images of skin burn wound healing at 0, 3, 5, and 7 days post skin burn wounding in mice treated with systemic saline (no-treatment) or with systemic 4-AP. Scale bar, 1 mm. FIGS. 1B and 1C) Graphs showing burn wound healing (FIG. 1B) and burn wound area (FIG. 1C) at each time point relative to the initial wound area in saline treated mice and in 4-AP treated mice. Data showed a significant burn wound protection from expansion of wound (protection from necrosis of skin burn at zone of stasis) and a significant increase in wound closure by day 3 in 4-AP treated compared to saline treated (control) animals (each value represents mean±SEM, n=9 animals per group, statistical significance indicated by asterisks (*=P between 0.01 and 0.05). Comparisons were performed using two-way ANOVA (Sidak's multiple comparisons test).

FIGS. 2A and 2B show morphometric wound healing assessments. FIG. 2A) Representative images of H&E staining of the saline control and 4-AP treated of day 26. FIG. 2B) Representative images of Masson's trichrome stained images of saline control and 4-AP treated wounds on day 26.

### DETAILED DESCRIPTION

This document provides methods and materials for treating burns. For example, compositions containing 4-AP and/or one or more derivatives of 4-AP can be used to treat a mammal (e.g., a human) having one or more burns. In some cases, a composition containing 4-AP and/or one or more derivatives of 4-AP can be administered (e.g., systemically administered) to a mammal (e.g., a human) having one or more burns to treat the mammal (e.g., to promote burn wound healing on the mammal).

In some cases, a composition described herein (e.g., a composition containing 4-AP and/or one or more derivatives of 4-AP) can be administered to a mammal (e.g., a human) in need thereof (e.g., a human having one or more burns) to accelerate burn wound healing on the mammal. For example, a composition described herein can be administered to a mammal having one or more burns to accelerate burn wound healing on the mammal by, for example, 10, 20, 30, 40, 50, 60, 70, 80, 90, 95, or more percent.

In some cases, a composition described herein (e.g., a composition containing 4-AP and/or one or more derivatives of 4-AP) can be administered to a mammal (e.g., a human) in need thereof (e.g., a human having one or more burns) to accelerate skin regeneration of a burn wound. For example, a composition described herein can be administered to a mammal having one or more burns to accelerate skin regeneration of a burn wound on the mammal by, for example, 10, 20, 30, 40, 50, 60, 70, 80, 90, 95, or more percent.

In some cases, a composition described herein (e.g., a composition containing 4-AP and/or one or more derivatives of 4-AP) can be administered to a mammal (e.g., a human) in need thereof (e.g., a human having one or more burns) to increase re-epithelialization at the burn wound(s). For example, a composition described herein can be administered to a mammal having a wound (e.g., a skin wound) to increase re-epithelialization at the burn wound(s) on the mammal by, for example, 10, 20, 30, 40, 50, 60, 70, 80, 90, 95, or more percent.

Any appropriate mammal can be treated as described herein. Examples of mammals that can be treated as described herein include, without limitation, humans, non-human primates such as monkeys, horses, bovine species, porcine species, dogs, cats, mice, rats, rabbits, and goats. In some cases, a human having one or more burns can be treated as described herein (e.g., by administering a composition containing 4-AP and/or one or more derivatives of 4-AP to the human).

A mammal having any type of burn can be treated as described herein (e.g., by administering a composition described herein). A burn can affect any part of a mammal (e.g., any part of a mammal's body). In some cases, a burn can be a skin (cutaneous) burn. In some cases, a burn can be an internal burn. A burn can be any degree (e.g., first-, second-, third-, or fourth-degree) of burn. Examples of burns that can be treated as described herein include, without limitation, thermal burns, flame burns, scalding burns, cold burns, chemical burns, electricity burns, radiation burns (e.g., ionizing radiation burns and non-ionizing radiation burns), and ultraviolet (UV; e.g., sun) burns.

In some cases, the methods described herein can include identifying a mammal (e.g., a human) as having a burn. Any appropriate method can be used to identify a mammal as having a burn. For example, visual inspection and/or physical examinations can be used to identify mammals (e.g., humans) as having a burn.

A mammal having one or more burns can be administered or instructed to self-administer a composition described herein (e.g., a composition containing 4-AP and/or one or more derivatives of 4-AP).

A composition described herein (e.g., a composition containing 4-AP and/or one or more derivatives of 4-AP) can be administered to a mammal in need thereof (e.g., a mammal having one or more burns) at any appropriate time. In some cases, a composition described herein can be administered within 7 days of the mammal sustaining one or more burns. For example, a composition described herein can be administered immediately after to within about 7 days of the mammal sustaining one or more burns. In some cases, a composition described herein can be administered within 7 days of the mammal being identified as having one or more burns. For example, a composition described herein can be administered immediately after to within about 7 days of the mammal being identified as having one or more burns.

A composition described herein (e.g., a composition containing 4-AP and/or one or more derivatives of 4-AP) can include 4-AP and/or any appropriate derivative(s) of 4-AP. Examples of derivatives of 4-AP that can be included in a composition described herein include, without limitation, 3,4-diaminopyridine, 3-hydroxy-4-aminopyridine, N-(4-pyridyl)-t-butyl carbamate, N-(4-pyridyl)ethyl carbamate, N-(4-pyridyl) methyl carbamate, and N-(4-pyridyl) isopropyl carbamate. In some cases, 4-AP and/or one or more derivatives of 4-AP can have a structure according to Formula I:

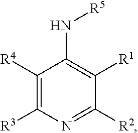

where R¹, R², R³, R⁴, and R⁵ are each independently selected from hydrogen (H), halogen (e.g., fluorine (F), chlorine (Cl), bromine (Br) or iodine (I)), amine, hydroxyl (e.g., —OH), alkoxy (e.g., —OAk), carboxyl (e.g., —CO₂H), alkoxycarbonyl (e.g., —C(O)—OAk), or alkyl (e.g., C₁₋₆ alkyl or Ak). For example, R¹, R², R³, R⁴, and R⁵ can all be hydrogen. Examples of amine include —NR^{N1}R^{N2}, in which each of R^{N1} and R^{N2} is, independently, H or optionally substituted alkyl, or R^{N1} and R^{N2}, taken together with the nitrogen atom to which each are attached, form a heterocyclyl group. Examples of alkyl or Ak include a branched or unbranched saturated hydrocarbon group of 1 to 24 carbon atoms (C₁₋₂₄), such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. Substitutions for alkyl can include a halogen (e.g., any herein), hydroxyl, alkoxy, carboxyl, or amine.

In some cases, 4-AP or a derivative thereof can be a potassium channel blocker. In some cases, 4-AP or a derivative thereof can be a calcium channel agonist. In some cases, 4-AP or a derivative thereof can be electrically active. In some cases, 4-AP or a derivative thereof can be in the form of a free base. In some cases, 4-AP or a derivative thereof can be in the form of a salt (e.g., pharmaceutically acceptable salt). When 4-AP or a derivative thereof is in the form of a salt, the salt can include any appropriate acid (e.g., an organic acid or an inorganic acid). Examples of acids that can be used to form a salt with 4-AP or a derivative thereof include, without limitation, hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, and mandelic acid.

In some cases, 4-AP and/or one or more derivatives of 4-AP can be as described elsewhere (see, e.g., U.S. Patent Application Publication No. 2018/0271847, U.S. Pat. No. 9,993,429, and International Patent Application Publication WO 2021/150773).

A composition described herein (e.g., a composition containing 4-AP and/or one or more derivatives of 4-AP) can include any appropriate amount of 4-AP and/or one or more derivatives of 4-AP. In some cases, a composition described herein can include from about 0.1 μM to about 150 μM (e.g., from about 0.1 μM to about 125 μM, from about 0.1 μM to about 100 μM, from about 0.1 μM to about 75 μM, from about 0.1 μM to about 50 μM, from about 0.1 μM to about 30 μM, from about 0.1 μM to about 20 μM, from about 0.1 μM to about 10 μM, from about 0.1 μM to about 5 μM, from about 0.1 μM to about 1 μM, from about 1 μM to about 150 μM, from about 5 μM to about 150 μM, from about 10 μM to about 150 μM, from about 20 μM to about 150 μM, from about 30 μM to about 150 μM, from about 50 μM to about 150 μM, from about 75 μM to about 150 μM, from about 100 μM to about 150 μM, from about 125 μM to about 150 μM, from about 1 μM to about 125 μM, from about 5 μM to about 100 μM, from about 10 μM to about 75 μM, from about 25 μM to about 50 μM, from about 5 μM to about 25 μM, from about 25 μM to about 50 μM, from about 50 μM to about 75 μM, from about 75 μM to about 100 μM, or from about 100 μM to about 125 μM) of 4-AP and/or one or more derivatives of 4-AP. In some cases, a composition described herein can include from about 0.01% to about 99% (e.g., from about 0.01% to about 90%, from about 0.01% to about 80%, from about 0.01% to about 70%, from about 0.01% to about 60%, from about 0.01% to about 50%, from about 0.01% to about 40%, from about 0.01% to about 30%, from about 0.01% to about 20%, from about 0.01% to about 10%, from about 0.01% to about 5%, from about 0.01% to about 1%, from about 1% to about 99%, from about 5% to about 99%, from about 10% to about 99%, from about 20% to about 99%, from about 30% to about 99%, from about 40% to about 99%, from about 50% to about 99%, from about 60% to about 99%, from about 70% to about 99%, from about 80% to about 99%, from about 90% to about 99%, from about 10% to about 90%, from about 20% to about 80%, from about 30% to about 70%, from about 40% to about 60%, from about 10% to about 30%, from about 30% to about 50%, from about 50% to about 70%, or from about 70% to about 90%) of 4-AP and/or one or more derivatives of 4-AP.

In some cases, a composition described herein (e.g., a composition containing 4-AP and/or one or more derivatives of 4-AP) can include one or more pharmaceutically acceptable carriers (additives), excipients, and/or diluents. Examples of pharmaceutically acceptable carriers, excipients, and diluents that can be used in a composition described herein include, without limitation, saline (e.g., phosphate-buffered saline (PBS)), sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition described herein (e.g., a composition containing 4-AP and/or one or more derivatives of 4-AP) can be administered to a mammal in need thereof (e.g., a mammal having one or more burns) locally or systemically. In some cases, a compositions described herein can be administered locally. For example, a composition described herein can be administered locally by injection directly into, around, and/or near a burn on a mammal (e.g., a human). For example, a composition described herein can be administered locally by topical administration directly to the skin (e.g., to a burn wound on the skin) onto, around, and/or near a burn on a mammal (e.g., a human). Compositions suitable for topical administration include, without limitation, creams, foams, gels (e.g., thermogels), lotions, ointments, and dressing materials (e.g., TEGADERM™). In some cases, a composition described herein can be administered systemically. For example, a composition described herein can be designed for oral or parenteral (including intraperitoneal, subcutaneous, intramuscular, intravenous, and intradermal) administration to a mammal having one or more burns. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition described herein can be formulated for parenteral administration (e.g., intravenous injection).

Any appropriate amount can be administered to a mammal to treat the mammal as described herein. For example, an effective amount of 4-AP and/or one or more derivatives of 4-AP that can be administered to a mammal (e.g., a human) can be any amount that can treat one or more burns in a mammal without producing significant toxicity to the mammal. In some cases, an effective amount of 4-AP and/or one or more derivatives of 4-AP can be from about 0.01 milligrams per kilogram body weight (mg/kg) to about 2 mg/kg (e.g., from about 0.01 mg/kg to about 1.8 mg/kg, from about 0.01 mg/kg to about 1.5 mg/kg, from about 0.01 mg/kg to about 1.3 mg/kg, from about 0.01 mg/kg to about 1 mg/kg, from about 0.01 mg/kg to about 0.7 mg/kg, from about 0.01 mg/kg to about 0.5 mg/kg, from about 0.01 mg/kg to about 0.2 mg/kg, from about 0.01 mg/kg to about 0.05 mg/kg, from about 0.05 mg/kg to about 2 mg/kg, from about 0.1 mg/kg to about 2 mg/kg, from about 0.5 mg/kg to about 2 mg/kg, from about 0.8 mg/kg to about 2 mg/kg, from about 1 mg/kg to about 2 mg/kg, from about 1.3 mg/kg to about 2 mg/kg, from about 1.5 mg/kg to about 2 mg/kg, from about 1.7 mg/kg to about 2 mg/kg, from about 0.05 mg/kg to about 1.7 mg/kg, from about 0.1 mg/kg to about 1.5 mg/kg, from about 0.5 mg/kg to about 1.2 mg/kg, from about 0.8 mg/kg to about 1 mg/kg, from about 0.5 mg/kg to about 0.8 mg/kg, from about 1 mg/kg to about 1.2 mg/kg, from about 1.2 mg/kg to about 1.5 mg/kg, or from about 1.5 mg/kg to about 1.8 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the burn may require an increase or decrease in the actual effective amount administered.

The frequency of administration of a composition described herein (e.g., a composition containing 4-AP and/or one or more derivatives of 4-AP) can be any frequency that can treat a burn in a mammal without producing significant toxicity to the mammal. For example, the frequency of administration can be from about four times a day to about once every other day, from about three times a day to about once every other day, from about twice a day to about once every other day, from about once a day to about once every other day, from about four times a day to about once a day, from about three times a day to about once a day, from about twice a day to about once a day, from about three times a day to about once every other day, or from about twice a day to about once a day. The frequency of administration can remain constant or can be variable during the duration of treatment. A course of treatment with a composition described herein can include rest periods. For example, a composition described herein can be administered once a month over a six-month period followed by a rest period (e.g., a one or two month rest period), and such a regimen can be repeated multiple times. As with the effective amount, various factors can influence the actual frequency of administration used for a particular application. For example, the effective amount, duration of treatment, use of multiple treatment agents, route of administration, and severity of the burn may require an increase or decrease in administration frequency.

An effective duration for administering a composition described herein (e.g., a composition containing 4-AP and/or one or more derivatives of 4-AP) can be any duration that can treat a burn in a mammal without producing significant toxicity to the mammal. For example, the effective duration can vary from several days to several weeks, months, or years. In some cases, the effective duration can be until a burn heals (e.g., until the wound healing appears complete). In some cases, the effective duration for the treatment of a burn can range in duration from about one month to about 6 months. Multiple factors can influence the actual effective duration used for a particular treatment. For example, an effective duration can vary with the frequency of administration, effective amount, use of multiple treatment agents, route of administration, and severity of the burn being treated.

In some cases, the methods and materials described herein can be used as the sole active agent used to treat a mammal (e.g., a human) having one or more burns. For example, a composition containing 4-AP and/or one or more derivatives of 4-AP can be used as the sole active agent(s) used to treat a mammal (e.g., a human) having one or more burns.

In some cases, methods described herein can include administering to a mammal (e.g., a mammal having one or more burns) one or more (e.g., one, two, three, or more) additional agents used to treat a burn (e.g., one or more agents in addition to a composition described herein such as a composition containing 4-AP and/or one or more derivatives of 4-AP). The one or more additional agents used to treat a burn can include any appropriate agent(s) used to treat a burn. In some cases, when treating a burn with 4-AP and/or one or more derivatives, additional agents can be included and/or used in combination with the 4-AP and/or one or more derivatives. For example, agents that treat infections, pain medications, and anti-anxiety medication can be used in combination with 4-AP and/or one or more derivatives when using 4-AP and/or one or more derivatives to treat a burn wound. Examples of additional agents that can be used in combination with the 4-AP and/or one or more derivatives include, without limitation, penicillin, erythromycin, amoxicillin, methicillin, chlorhexidine gluconate, chloroxylenol, hydrogen peroxide, iodine, opioid analgesics (e.g., morphine), non-steroidal anti-inflammatory drugs (NSAIDs; e.g., ibuprofen), naproxen sodium, acetaminophen, midazolam, propofol, and silver sulfadiazine. In cases where a mammal having one or more burns is treated with a composition described herein and is treated with one or more additional agents (e.g., one or more addition agents used to treat a burn and/or one or more additional agents used to treat infection, pain, and/or anxiety), the additional agent(s) can be administered at the same time or independently. For example, the additional agent(s) can be formulated into a composition containing 4-AP and/or one or more derivatives of 4-AP to form a single composition. In some cases, a composition described herein can be administered first, and the one or more additional agents can be administered second, or vice versa.

In some cases, methods described herein can include subjecting a mammal (e.g., a mammal having one or more burns) to one or more (e.g., one, two, three, or more) therapies used to treat a burn (e.g., one or more agents in addition to a composition described herein such as a composition containing 4-AP and/or one or more derivatives of 4-AP). Examples of therapies that can be used to treat a burn include, without limitation, intravenous (IV) fluids (e.g., to prevent dehydration and organ failure), skin grafts, and plastic surgery. In cases where a mammal having one or more burns is treated with a composition described herein and is subjected to one or more therapies used to treat a burn, the one or more therapies can be performed at the same time or independently of the administration of the composition described herein. For example, a composition containing 4-AP and/or one or more derivatives of 4-AP can be administered before, during, or after the one or more therapies are performed.

In some cases, the healing of one or more burns present on a mammal being treated can be monitored. Any appropriate method can be used to determine the healing of the burn(s) present on a mammal. For example, visual inspection, and/or physical examinations can be used to assess the healing of one or more burns present on a mammal.

The invention will be further described in the following examples, which do not limit the scope of the invention described in the claims.

### EXAMPLES

### Example 1: Burn Wound Healing with 4-Aminopyridine

This Examples describes using 4-AP to treat burns.

### Methods

Place the 8-10-week-old wild-type C57BL/6 mouse on its side on a sterile paper sheet. Anesthesia was achieved and maintained with isoflurane utilizing an induced with an intraperitoneal injection of ketamine (60 mg/kg) and xylazine (4 mg/kg). An adequate depth of anesthesia was ensured via lack of a withdrawal reflex when a toe pinch was performed. The depth of anesthesia was monitored as well as respiration and mucous membrane color (pink: normal, pale: blood loss, blue: cyanosis). The surgeon(s) used sterile gloves, mask and a clean lab coat. Sterile surgical instruments (autoclaved) were used during all survival surgeries. The skin was prepared for creation of wound on the dorsal and side skin (from neck to tail) by shaving with a mechanical shaver and then applying a hair removal cream on the skin for 3-5 minutes. Hair was removed completely by gently wiping the skin with cotton balls soaked in warm water. Skin disinfection around the incision site was done using 70% ethanol and betadine for 3 times. Then, the dorsal skin was folded and raised cranially and caudally at the midline using index fingers and thumbs, and the animal was placed in a lateral position. The burn wound was created by contacting a 10 mm diameter brass heating device maintained at 100° C. to the mouse dorsum at a constant pressure. The device remained in contact with the skin for 4 seconds in order to create a deep-partial thickness burn. After burn wound creation, the wound site was photographed, the wound area was circled using permanent pen marker, and the wound surface was covered with a TEGADERM™ (3M) sterile transparent dressing. After surgery, mice were given SR Buprenorphine (0.05 mg/kg) for post-operative analgesia. Then, mice were placed in a clean cage on the heating pad for 30 minutes to help the recovery process. Immediately after the recovery, mice were randomly grouped and treated with either with 4-AP (systemic, ip) or saline (control).

Functional analysis of the burn wound area was performed by placing a transparent sheet on the surface of the circled wound, and placing a ruler close to the burn wound to capture photographs of individual wounds on 0, 3, 7, 10, 14, 18, 21, and 26 days. The wound size of each individual mice was measured and compared to zero day wound diameter from the circled sheet. The percentage of wound closure was calculated using this formula: (area of original wound-area of the actual burn wound)/area of original burn wound×100.

Burn wound healing assessments were conducted at 26 day post wound using formalin-fixed paraffin-embedded tissue that serially sectioned into 5-μm thick sections on a Microtome. Skin sections were processed for morphometric analysis using hematoxylin and eosin (H&E) and Masson's trichrome stain stained tissues were imaged by light microscopy.

### Results

4-AP can promote burn wound closure and tissue regeneration.

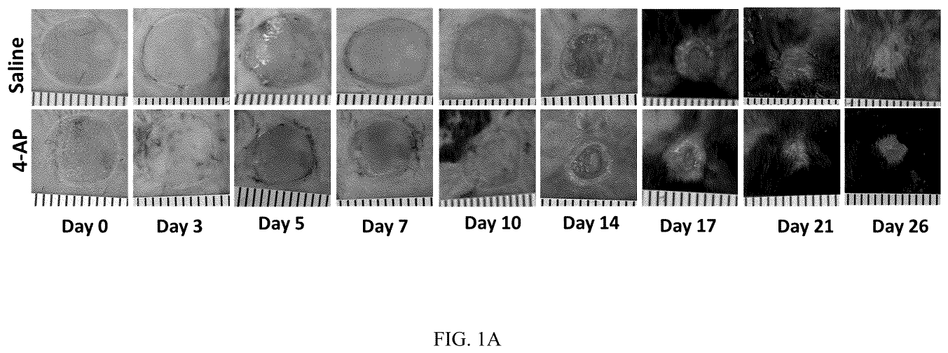

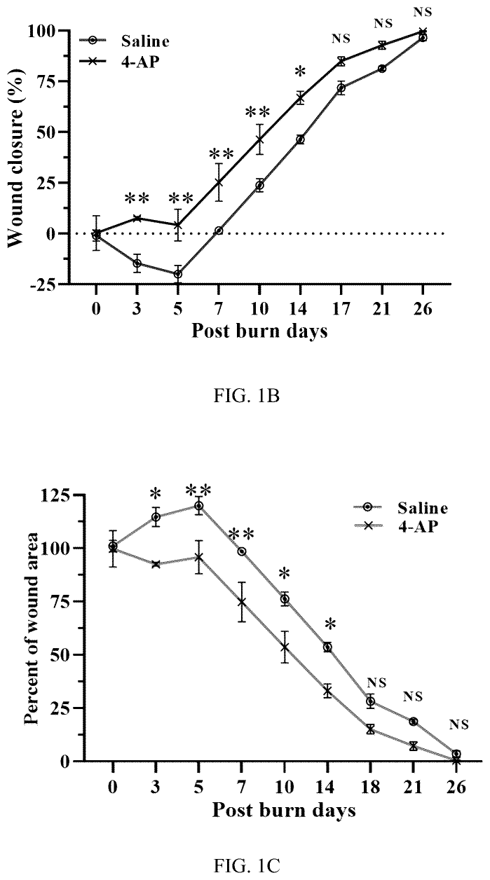

Mice having burn wounds were treated with systemic 4-AP or systemic saline (control animals). Mice having burn wounds that were treated with systemic 4-AP demonstrated improved burn wound closure and tissue regeneration as compared to control animals (e.g., mice having burn wounds and administered systemic saline). Representative images of wounds are shown in FIG. 1A. Rejuvenated skin cells can be seen at the zone of stasis and accelerate skin burn wound healing and regeneration. Systemically administered 4-AP treatment significantly prevented expansion of wound size from necrosis and apoptosis. Necrosis of burn skin cells at the zone of stasis showed a 25% expansion of wound area in saline treated animals (n=125) and 5% reduced wound area in 4-AP treated animals (n=95) on day 5. Burn wound closure is shown in FIG. 1B. The percent of wound closure is shown in FIG. 1C. Treatment with 4-AP protected burn wounds from expansion (from necrosis), accelerated wound closure by day 3, and resulted in closed wounds by day 26. These data show that systemically administered 4-AP can accelerate skin burn wound healing and regeneration.

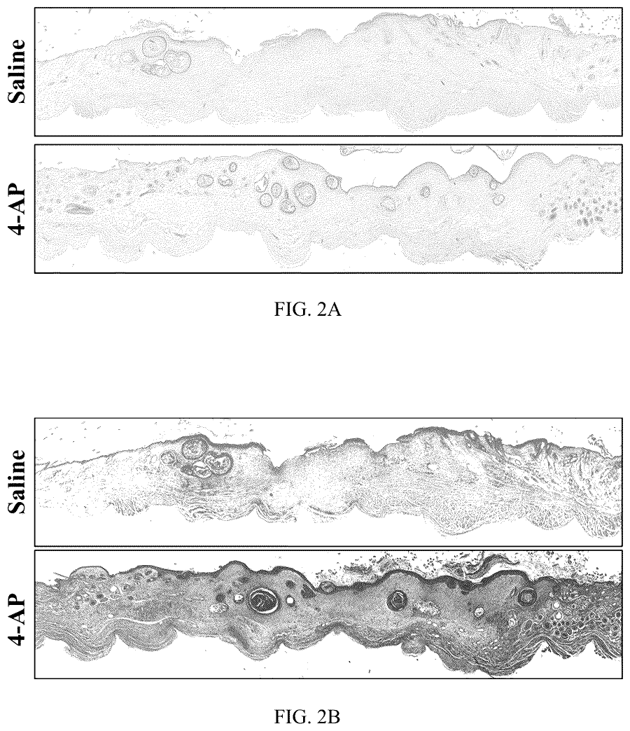

The fidelity of 4-AP-induced burn wound healing was further assessed at day 26, when re-epithelialization was complete. Wound tissue was analyzed by morphometric analysis on hematoxylin and eosin (H&E) stained sections, which revealed newly formed epidermis within the healed wound that was significantly thicker in 4-AP treated mice while in saline re-epithelization was not completed in saline-treated mice (FIG. 2A). To test whether 4-AP treatment affects fibroblast maturation during burn wound healing, Masson's Trichrome staining to measure collagen deposition in the healing wound was performed. Staining revealed elevated collagen deposition in 4-AP treated mice compared to saline-treated mice (FIG. 2B).

These results demonstrate that 4-AP can be used to treat mammals having burns.

### Example 2: Treating Burns

A human identified as having a burn is systemically administered a composition containing 4-AP and/or one or more derivatives of 4-AP. The administered composition can be effective to heal the burn (e.g., to heal the burn to a greater extent than in the absence of the composition containing 4-AP and/or one or more derivatives of 4-AP).

### Example 3: Promoting Burn Wound Healing

A human identified as having a burn is systemically administered a composition containing 4-AP and/or one or more derivatives of 4-AP. In some cases, the administered composition can promote healing (e.g., closure) of a burn wound. In some cases, the administered composition can reduce the size of a burn wound (e.g., to reduce the size of the burn to a greater extent than in the absence of the composition containing 4-AP and/or one or more derivatives of 4-AP).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a burn, wherein said method comprises administering a composition comprising 4-AP or one or more derivatives of said 4-AP to a mammal identified as having said burn.

2. A method for promoting burn wound healing, wherein said method comprises administering a composition comprising 4-AP or one or more derivatives of said 4-AP to a mammal identified as having a burn, thereby promoting healing of said burn.

3. A method for accelerating skin regeneration of a burn region, wherein said method comprises administering a composition comprising 4-AP or one or more derivatives of said 4-AP to a mammal identified as having a burn, thereby accelerating skin regeneration of tissue around said burn.

4. The method of claim 1, wherein said mammal is a human.

5. The method of claim 1, wherein said burn is a cutaneous burn.

6. The method of claim 1, wherein said burn is selected from the group consisting of a thermal burn, a flame burn, a scalding burn, a cold burn, a chemical burn, an electricity burn, an ionizing radiation burn, a non-ionizing radiation burn, and a sun burn.

7. The method of claim 1, wherein said administration is a systemic administration.

8. The method of claim 1, wherein said administration is a topical administration.

9-14. (canceled)
